(19) United States
(12) Patent Application Publication
(10) Pub. No.: US 2023/0405068 A1
(43) Pub. Date: Dec. 21, 2023

(54) RADIATION-INDUCED FIBROSIS TREATMENT

(71) Applicant: Handelsselskabet af 20.10.2020 ApS, Havdrup (DK)

(72) Inventor: Jens Steen OLSEN, Havdrup (DK)

(73) Assignee: Handelsselskabet af 20.10.2020 ApS, Havdrup (DK)

(21) Appl. No.: 18/035,432

(22) PCT Filed: Nov. 8, 2021
(86) PCT No.: PCT/EP2021/080897

§ 371 Date: May 4, 2023

(30) Foreign Application Priority Data
Nov. 6, 2020 (DK) PA202070739

(51) Int. Cl. A61K 36/48; A61K 35/02; A61P 39/00

(52) U.S. Cl. CPC A61K 36/48; A61K 35/02; A61P 39/00

(57) ABSTRACT

Disclosed herein is a pharmaceutical composition for use in the treatment or prevention of radiation-induced fibrosis, wherein the composition comprises an extract of Trigonella foenum-graecum and optionally pharmaceutically acceptable additives.

### TECHNICAL FIELD

The present invention relates to a composition for use in the treatment of radiation-induced fibrosis.

### BACKGROUND

Radiation is used for the treatment of a wide range of cancers. Since radiation must penetrate the skin to reach the tumor site, the skin receives dose-dependent damage during radiation treatment. The skin is susceptible to radiation damage because it is a continuously renewing organ, which contains rapidly proliferating and maturing cells, with basal keratinocytes, hair follicle stem cells and melanocytes being very radiosensitive.

The most sensitive skin areas are the anterior of the neck, extremities, chest, abdomen and face, along with the hair follicles on the scalp and breast tissue.

The skin injury manifests itself as radiation-induced dermatitis in approximately 95% of patients receiving radiation exposure, with the injury ranging from mild erythema to moist desquamation and ulceration. Radiation-induced dermatitis is an acute skin reaction and can lead to pain, itching, poor aesthetic appearance and delays in radiation treatment. In the long term, skin wounds can reappear due to abnormal pathological changes, such as excessive fibrosis that can occur during the initial phases of the healing process. The acute and delayed effects of radiation therapy to skin can decrease quality of life for many patients.

The most common strategy for preventing and minimizing radiation-induced dermatitis involve: simple moisturization of the irradiated area, use of a mild soap to keep the area clean and avoidance of potential mechanical irritants such as scratching and rough clothing. However, the currently used treatment regimens lack clinically significant efficacy. Washing with a mild soap had no effect on erythema score or mean time to maximal toxicity. Other treatments, such as the use of aloe vera gel, hyaluronidase-based creams or sucralfate creams, do not result in significant improvements in dermatitis scoring. Overall, the clinical trials evaluating a large assortment of products and methods for the prevention of radiation-induced dermatitis do not support a general consensus on an effective treatment.

Administration of antioxidants or anti-inflammatory compounds alleviates the oxidative stress caused by radiation. For example, intramuscular injection of Sod1 (Cu—Zn) protein protected both humans and pigs from radiation-induced skin fibrosis. Moreover, the antioxidant aminothiols amifostine and glutathione limit mitochondrial lipid peroxidation and protect from radiation-induced injury. The thiol antioxidant and glutathione precursor N-acetylcysteine also significantly protects rats from radiation-induced dermatitis. In a clinical trial, topical corticosteroid (0.1% methylprednisolone) treatment ameliorated but did not prevent radiation-induced dermatitis, suggesting that more potent anti-inflammatory interventions or the combined effect of antioxidants and anti-inflammatory agents may be necessary for prevention and/or treatment of radiation-induced dermatitis.

Synthetic triterpenoids are hypothesized to be effective against radiation-induced dermatitis because of their dual roles in alleviating both oxidative stress and inflammation. RTA 408 (omaveloxolone) is a synthetic oleanane triterpenoid allegedly protecting mice from radia-induced dermatitis when applied topically, Scott A. Reisman; Chun-Yue I. Lee; Colin J. Meyer; Joel W. Proksch; Stephen T. Sonis; Keith W. Ward, Radiat Res (2014) 181 (5): 512-520. However, the lotion has never been approved for marketing.

Radiation-induced fibrosis (RIF) is a long-term side effect of external beam radiation therapy for the treatment of cancer. It results in a multitude of symptoms that significantly impact quality of life. Radiation-induced fibrosis may cause both cosmetic and functional impairment, which can lead to death or significant deterioration in quality of life. Developing effective strategies to prevent long-term disability and discomfort following radiation therapy have been a priority for many companies and research groups.

Radiation-induced fibrosis can develop as a late effect of radiation therapy in skin and subcutaneous tissue, the lungs, the gastrointestinal and genitourinary tracts, muscles, or other organs, depending upon the treatment site. The development of radiation-induced fibrosis is influenced by multiple factors, including radiation dose and volume, fractionation schedule, previous or concurrent treatments, genetic susceptibility, and comorbidities such as diabetes mellitus. Although radiation-induced fibrosis originally was assumed to be a slow, irreversible process, contemporary studies suggest that it is not necessarily a fixed process.

Currently, there are no approved agents for the prevention of radiation-induced fibrosis. Thus, a high unmet medical need exists for providing a pharmaceutical composition useful in the prevention or treatment of radiation-induced fibrosis.

### SUMMARY

It is an object to provide a pharmaceutical composition for use in the treatment or prevention of radiation-induced fibrosis, wherein the composition comprises an extract of Trigonella foenum-graecum and optionally pharmaceutically acceptable additives.

The present inventor unexpectedly discovered that the pharmaceutical composition, when applied to an area of the skin or mucous membrane having received radiation therapy, resulted in little or no dermal or mucous fibrosis.

In an implementation of the pharmaceutical composition the further compound bentonite was added. In some formulations of the pharmaceutical composition the mixture of the extract of Trigonella foenum-graecum and bentonite behaves synergistically, i.e. the effect of the extract is potentiated by the presence of bentonite.

In a certain implementation of the pharmaceutical composition the bentonite comprises 50% by weight or more of smectite. Suitably, the pharmaceutical composition comprises 1:10 to 10:1 by weight dry matter of Trigonella foenum-graecum extract to bentonite. In an embodiment of the invention the weight of Trigonella foenum-graecum extract in the pharmaceutical composition is at least 0.01% by weight of the pharmaceutical composition.

The pharmaceutical composition of the invention may formulated in any way suitable for the path of administration. In a certain implementation of the pharmaceutical composition, it is formulated as a gel, cream, plaster, spray, ointment, throat lozenge, or tablet. Generally, when the pharmaceutical composition is administrated topically, it is formulated as a gel, cream, plaster, or ointment. When the pharmaceutical composition is administrated to a mucous membrane it is generally in the form of a spray or at throat lozenge. A spray is usually preferred for administration to a mucous membrane of the lung, whereas a throat lozenge is a suitable administration form when the radiation-induced fibrosis is mucosal fibrosis, such as mucosal fibrosis present in the throat.

According to an implementation of the pharmaceutical composition the throat lozenge is sublingual or buccal administered. Suitably, the throat lozenge administered sublingual is dissolved during a time period of 1 to 20 minutes.

Generally, when the radiation-induced fibrosis is dermal fibroses, the pharmaceutical composition is formulated as a gel, cream, ointment or spray.

In a certain implementation of the pharmaceutical composition, the radiation therapy includes external beam radiation.

The start of the treatment or the preventive treatment may be initiated any suitable time in relation to the radiation therapy, i.e. the treatment or preventive treatment may be initiated prior to, during or after the radiation therapy. In a certain implementation of the invention the preventive treatment of radiation-induced fibrosis is initiated during the radiation therapy. Treatment early in the radiation therapy, such as within 1, 2, or 3 weeks after the initiation of the radiation therapy, has shown to be effective and furthermore resulted in a reduced tendency of the formation of radiation-induced dermatitis.

A pronounced effect in terms of reducing the radiation-induced fibrosis may also be obtained when the treatment with the pharmaceutical composition according to the present invention is initiated after the end of the radiation therapy. Suitably, the treatment of radiation-induced fibrosis is initiated within two years after the end of the radiation therapy.

In a certain implementation of the present invention, the prevention or treatment of radiation-induced dermal fibrosis comprises application of the pharmaceutical composition at least one time daily on the skin tissue having received radiation. Suitably, the pharmaceutical composition is applied more frequently, such as 2, 3, 4 or more times daily. Similarly, when radiation-induced mucosal fibrosis is treated or prevented by the present pharmaceutical composition, the mucosal membrane may be subjected to the pharmaceutical composition 1, 2, 3, 4 or more times daily.

In an embodiment of the pharmaceutical composition the duration of the treatment is at least one week. Suitably, the treatment or preventive treatment extends over several weeks such as 2, 3, 4, 5, 6 weeks or more.

The pharmaceutical composition according to the present invention may be used to alleviate or treat the side effects of radiation therapy when a multitude of cancer types are treated. In a certain implementation, the disease treated with radiation therapy is throat cancer, breast cancer, lung cancer, melanoma cancer, testis cancer, or head and neck cancer.

The Trigonella foenum-graecum extract may be obtained by any method known by the person skilled in the art for extracting components from plant materials. In an implementation of the invention the extract is obtainable by the steps of: preparing a mixture of seeds from Trigonella foenum-graecum and liquid, incubating said mixture for at least 3 hours, heating of said mixture at least until the embryo is released from the seeds, and recovering a liquid extract from mixture e.g. by separating remaining plant material from the mixture.

Suitably, the incubation in step b) is continued until the sprouting is visible. The method for preparing the extract may further comprise the step of removing the solvent by spray drying. Thus, the Trigonella foenum-graecum extract in the pharmaceutical composition may be spray dried particles.

### DETAILED DESCRIPTION

### Extract

Trigonella foenum-graecum (also termed Fenugreek or TFG herein) is an annual herb belonging to the legume family. The TFG seed is a major constituent of curry and a part of traditional Indian and Asian cooking. TFG is considered safe as a human food component, taste enhancer, and coloring agent. The TFG seed are rich in phytochemicals, including proteins, steroidal saponin, flavanoids, tannic acids, stearic acid, vegetal oils, alkaloide trigonelline and 4-hydroxyisoleucine.

A method for preparation of an extract from Trigonella foenum-graecum may be found in WO 08/125120. According to the disclosure of the prior art publication, seeds of Trigonella foenum-graecum are submerged in water to initiate sprouting before the extraction. WO 17/207010 discloses a composition comprising a mixture of Trigonella foenum-graecum extract and bentonite. WO 08/125120 and WO 17/207010 are incorporated herein by reference.

In one aspect of the invention the extract of trigonella foenum-graecum is derived from plant material, such as leaves, branches, flowers, seeds etc. In a suitable embodiment said plant material is seeds obtained from Trigonella foenum-graecum.

The method of preparing said extract may comprise the following steps:
a. preparing a blend of plant material and liquid,
b. incubating said blend for at least 3 hours,
c. heating of said blend,
d. recovering a liquid extract from blend eg. by separating remaining plant material from the blend.

The plant material may be fresh, frozen, dried, or combinations thereof. In the preferred embodiment the plant material is seeds of Trigonella foenum-graecum, most preferably dried seeds of said plant.

In order to facilitate the extraction of the active ingredients of the plant material, said plant material is soaked in a liquid, preferably water. The blend of liquid and plant material is incubated for at least 3 hours, more preferably at least 6 hours, preferably at least 12 hours, such as at least 24 hours. The incubation is usually performed at temperatures between 0 and 45° C., suitably at temperatures between 10 and 40° C. The incubation should preferably continue at least until the sprouting is visible.

Subsequently, the blend comprising the plant material soaked in a liquid is heated, preferably to a temperature above the coagulation of proteins. In a certain aspect the blend is boiled.

The blend comprises plant material and a liquid. The ratio by weight of said plant material and said liquid in said blend is suitably 1 to 1, or preferably less plant material by weight such as 1 to 2, or less plant material by weight such as 1 to 3, or less plant material by weight such as 1 to 4, or less plant material by weight such as 1 to 5, or less plant material by weight such as 1 to 6, or less plant material by weight such as 1 to 7, or less plant material by weight such as 1 to 8, or less plant material by weight such as 1 to 9, or less plant material by weight such as 1 to 10. In a preferred embodiment the ratio by weight of said plant material and said liquid is 1 to 6.

During the heating of the blend additional liquid may be added at least once in order to compensate for evaporated liquid and liquid taken up by the plant material. The liquid is heated for at least 5 minutes, such as 10 to 45 minutes, more preferably 20 to 30 minutes, such as 20 minutes. The heating may be terminated when the embryo is released from the seeds, which is associated with increased viscosity of the blend. Suitably, the heating is not continued more than minutes after the embryo has been released.

In one embodiment, the blend is frozen (preferably at −18° C.) prior to or after the heating step for at least 3 hours, preferably more than 6 hours, such as 12 hours, or more than 12 hours. Subsequently, the blend may be subjected to a second round of heating before recovery of the extract, e.g. by removing the remaining plant material. The freezing step is anticipated further to enhance the release of the active ingredients from the plant material.

The volume of a final concentrated extract originating from ½ kg of plant material such as seeds is approximately 2 liters.

For conservation the extract may be refrigerated. Depending on the application the extract may be diluted in water or used as it is. The extract may be further concentrated by removal of solvent. The solvent may be removed or reduced by any appropriate means, such as membrane filtration, evaporation, precipitation, extraction, azeotrope distillation, lyophilisation, spray drying and combinations thereof. The spray dried particles may subsequently be post-dried in a fluid bed apparatus.

The aqueous extract of Trigonella foenum-graecum has a tendency to smell of sotolon. The smell may be considered unpleasant by some users and may therefore restrict the application of the extract. The present inventors have surprisingly found that the amount of sotolon may be reduced drastically by spray drying the extract. Therefore, in a preferred aspect of the invention, the aqueous extract is dried by spray drying.

The extract used in the invention may be purified to isolate the active ingredient(s) by any appropriate method. Thus, the extract may be fractioned using gel filtration, HPCL, extraction, precipitation, etc. In a presently useful method, the extract is fractioned using HPLC. In a specific method the active ingredient(s) is included in an extract fraction obtainable by performing reverse phase chromatography on a size B Lichroprep RP-18 (40-631 μm) (Merck) of the basis extract using the following gradient: 0-1 min H₂O/AcN 98:2, then using a steady gradient from 1-40 min going to 100% and collecting the fraction at the time interval between 5 and 10 min. The solvent of the purified extract may be removed or reduced by any of the methods disclosed above. In an aspect of the invention, the purified extract is spray dried.

In another specific purification method, the aqueous extract may initially be treated with ethanol to precipitate the majority of the plant residues and polysaccharides from the extract. The precipitate may be removed by sedimentation or centrifugation. For easier storage, the solvent may be evaporated or otherwise removed so as to produce a powder. Alternatively, the ethanol treated extract may be used directly in the subsequent process. The powder may subsequently be suspended in water and acidified to pH 1-4, preferably pH 2, with a strong acid such as hydrochloric acid. The acidified extract is extracted with an organic water immiscible solvent like heptane. After agitation the organic and the aqueous layer are separated and the aqueous layer is treated with an alkaline agent to obtain a pH above pH 9, preferably around pH 10. The alkaline aqueous phase is again extracted with an organic water immiscible solvent and agitated. A solid powder is obtained from the recovered organic phase by removing the solvent by evaporation, such as by evaporation under reduced pressure or spray drying.

### Bentonite

Bentonite is an absorbent aluminum phyllosilicate clay. Bentonite is usually formed from weathering or diagenesis of volcanic ash. The different types of bentonite are generally named after the dominant ion, i.e. sodium bentonite, potassium bentonite, aluminum bentonite, iron bentonite, and calcium bentonite. Sodium and calcium bentonite are generally preferred.

Sodium bentonite is characterized by a high swelling power, often absorbing as much as several times its dry mass in water. Calcium bentonite is an adsorbent of ions and other components and is characterized by a somewhat lower swelling power. Calcium bentonite may have the calcium ion ex-changed with the sodium ion to convert it to sodium bentonite (termed sodium beneficiation or sodium activation) to exhibit many of sodium bentonite's properties by an ion exchange process. This transformation can be accomplished by adding a soluble sodium compound to the Ca-bentonite.

As bentonite is a naturally occurring clay, it may contain a complex blend of components. In general, the bentonite of the present invention comprises smectite clay. The smectite may e.g. be selected among montmorillonite, beidellite, sauconite, stevensite hectorite, saponite, nontronite, vermiculite, and mixtures thereof. Also present in the bentonite clay may be kaolin, illite, and/or chlorite. The amount of smectite in the bentonite is generally above 50% by weight, such as above 70% by weight, and suitably above 80% by weight.

Smectite is defined in clay mineralogy as a 2:1 clay—consisting of an octahedral sheet sandwiched between two tetrahedral sheets. Smectites are comprised of layers of negatively charged aluminosilicate sheets held together by charge-balancing counter-ions such as Na⁺ and Ca²⁺. In the presence of water these cations tend to hydrate, thereby forcing the clay layers apart in a series of discrete steps. This causes the smectite to swell.

It will be understood that the bentonite used in the composition of the pre-sent invention may be naturally occurring and unmodified bentonite, or any fraction thereof enriched in a certain component and optionally chemically modified, especially by exchange of ions. Montmorillonite is hydrated sodium calcium aluminum magnesium silicate hydroxide having the formula (Na,Ca)0.33(Al,Mg)2(Si4010) (OH)2·nH20. In one embodiment the bentonite clay is montmorillonite.

In aqueous environments, each smectite particle is composed of a multitude of submicroscopic platelets stacked in sandwich fashion with a layer of water between each. A single platelet is about one nanometer thick and up to several hundred nanometers across. Once the clay is hydrated, the weakly positive platelet edges are attached to the negatively charged platelet faces. A three-dimensional colloidal structure forms, which accounts for the characteristic rheology imparted by these clays, i.e. an increase in viscosity.

The amount of smectite in the composition is generally not above 50% by weight. For most practical purposes, an amount of approximately 30% by weight or less of bentonite clay is used to avoid a viscosity, which is not desirable from an end-user point of view. Suitably, the amount of smectite is 10% by weight or less, such as 5% by weight or less. To obtain a microorganism reducing effect even a small amount of bentonite is suitable. In general, the amount of smectite is 0.0001% by weight or above, such as 0.001% by weight, 0.01% by weight, 0.1% or above and preferably 0.3% by weight and above.

Bentonite clays are commercially available under various trade names including Van Gel B, Veegum, Veegum F, Veegum HV, VeegumK, Veegum HS, Veegum Ultra, Veegum D, Veegum Pure, Veegum Ultra, Veegum PRO, Veegum plus, Veegum T, Van Gel B, Van Gel C, Van Gel ES, Van Gel O, all trademarks of R.T. Vandebilt Company. Bentonite clays are also available from Amcol International.

Smectite clay may also be provided synthetically e.g. following the method of Nakazawa, H., Yamada, H., and Fujita, T. (1992): Crystal synthesis of smectite applying very high pressure and temperature, Applied Clay Science, 6, 395-401.

Bentonite occurs in many geological areas of the world. According to British Geological Survey bentonite is produced in at least 44 countries. Thus, sodium bentonite is i.a. produced in USA, in South Dakota and in Wyoming. Sodium bentonite is also produced in Turkey in the Tokat Resadiye region. Mixed sodium/calcium bentonite is mined in Greece, Australia, India, Russia and Ukraine. Calcium bentonite is mined in Mississippi and Alabama, Germany, Greece, Turkey, India, and China. In a certain aspect of the invention a bentonite produced in Denmark near Rødby is preferred.

The Trigonella foenum-graecum extract and bentonite may be mixed in any proportion that provide the intended effect. In a certain aspect of the invention the Trigonella foenum-graecum extract to bentonite is mixed in a weight ratio between 1:10 to 10:1. Suitably, the weight ratio between Trigonella foenum-graecum extract to bentonite is at least 2:10, such as 3:10. Similarly, the weight ratio of bentonite to Trigonella foenum-graecum extract is preferably at least 2:10, such as 3:10. In a preferred aspect the weight ratio between Trigonella foenum-graecum extract to bentonite is between 4:10 and 10:4.

### Formulation

The composition may comprise a variety of further components in addition to the Trigonella foenum-graecum extract and the bentonite for making up the final formulation. According to a certain aspect of the invention the weight of the mixture of Trigonella foenum-graecum extract and optionally bentonite is at least 0.01% by weight of the final formulation, such as at least 0.05% by weight and suitably at least 0.1% by weight.

The pharmaceutical composition according to the invention may be formulated in a number of different manners, depending on the purpose of the particular medicament and the type of administration. It is well within the scope of a person skilled in the arts to formulate compositions that are in accordance with the preferred type of administration.

The composition comprising the extract and optionally bentonite according to the invention may be prepared by any conventional technique, e.g. as described in Remington: The Science and Practice of Pharmacy 1995, edited by E. W. Martin, Mack Publishing Company, 19th edition, Easton, Pa.

The composition may comprise pharmaceutical acceptable additives such as any conventionally used pharmaceutical acceptable additive, which should be selected according to the specific formulation, intended administration route etc. For example, the pharmaceutically acceptable additives may be any of the additives mentioned in Nema et al, 1997. Furthermore, the pharmaceutical acceptable additive may be any accepted additive from FDA's “inactive ingredients list”, which can be downloaded from https://www.fda.gov/drugs/drug-approvals-and-databases/inactive-ingredients-database-download.

One preferred embodiment of the present invention is to provide a composition, such as a pharmaceutical or cosmetic composition, formulated for topical application on a local, superficial and restricted area such as a wound caused by radiation treatment, an area with radiation dermatitis, or an area intended for radiation therapy. According to the present invention, the term topical administration includes mocusal administration.

In said above-mentioned embodiment, the composition may be formulated as an ointment, a lotion, a crème, a gel, a paste, a milk, a suspension, an aerosol, a spray, a film, a foam, a serum, a swab, a pledget, a pad, a patch, a powder, a paste, a liniment, viscous emulsion, porridge, liquid, or another formulation which is appropriate for topical administration.

Such compositions for topical administration may further include physiologically acceptable components such as carriers, surfactants, preservatives, stabilizing agents, buffers, excipients and emulsifiers suited for this type of administration. Suitable components for topical delivery systems are preferably chosen from components that do not cause excessive or unavoidable irritation or pain to the recipient. Carriers include diluents and provide the medium in which the pharmaceutical constituents are dissolved, dispersed or distributed.

The composition according to the invention may comprise, but are not restricted to, a carrier such as an aqueous liquid base, nonaqueous liquid base, water soluble gel, a mineral oil base, emulsion, ointment, crème, gel or lotion, suspension of solid particles in a liquid.

The composition of the invention may be applied to skin and the active component(s) may exert their action on the skin or after penetration of the skin. The topical availability of active compounds depend on various factors including their ability to dissolve in the carrier (gel, cream—hydrophilic), and their ability to permeate the skin barrier (i.e., the stratum corneum—hydrophobic), thus requiring a certain hydrophobic-hydrophilic balance. Formulations may require addition of excipients, such as permeation enhancers and solubilizers to facilitate either or both of the transport processes (dissolution into vehicle and diffusion across skin). Additives, such as alcohols, fatty alcohols, fatty acids, mono- di- or tri-glycerides, glycerol monoethers, cyclodextrin and derivatives, polymers, bioadhesives, terpenes, chelating agents and surfactants have been disclosed to increase transdermal delivery of drugs. Alcohols include, but are not limited to, ethanol, 2-propanol and polyols such as polyethylene glycol (PEG), propylene glycol, glycerol, propanediol. It is within the present invention to make use of such excipients.

Any method, not limited to the above-mentioned, for increasing transdermal or transmucosal delivery is within the scope of the present invention. The medicament according to the present invention may therefore comprise surfactants such as ionic and/or non-ionic surfactants. Suitable non-ionic surfactants include for example: fatty alcohol ethoxylates (alkylpolyethylene glycols); alkylphenol polyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (Pluronic); carboxyvinyl polymer (Polygel® HP), fatty acid alkylolamides (fatty acid amide polyethylene glycols); alkyl polyglycosides, N-alkyl-, N-alkoxypolyhydroxy fatty acid amide, in particular N-methyl-fatty acid glucamide, sucrose esters; sorbitol esters, esters of sorbitol polyglycol ethers and lecithin. Ionic surfactants include for example sodium lauryl sulfate, sodium laurate, polyoxyethylene-20-cetylether, Laureth-9, sodium dodecylsulfate (SDS) and dioctyl sodium sulfosuccinate.

Methods for enhancing drug delivery through topical administration may be applied with the present invention, and include any means of increasing absorption, minimizing metabolism, and/or prolonging the half-life of the active ingredient of the medicament such as the extract of Trigonella foenum-graecum. Such means include the use of transporters of the type liposomes, ISCOMs, nano-particles, microspheres, hydrogels, organogels, polymers or other micro-encapsulation techniques.

In an embodiment of the invention in which the pharmaceutical composition is formulated as a gel, cream, lotion, ointment etc. one or more of the following additives may be used in the formulation: ethanol, allantoin, ammonia solution, anhydrous citric acid, ascorbic acid, benzalkonium chloride, benzoic acid, benzyl alcohol, betadex, boric acid, butylated hydroxyanisole, C13-14 isoparaffin/laureth-7/polyacrylamide, arbomer copolymer type b (allyl pentaerythritol crosslinked), carbomer homopolymer, carbomer homopolymer type a (allyl pentaerythritol crosslinked), carbomer homopolymer type b (allyl pentaerythritol crosslinked), carbomer homopolymer type b (allyl sucrose crosslinked), carbomer homopolymer type c (allyl pentaerythritol crosslinked), carboxymethylcellulose sodium, castor oil, citric acid monohydrate, cocoyl caprylocaprate, collagen, cyclomethicone/dimethicone copolyol, d&c yellow no. 10, denatonium benzoate, diethylene glycol monoethyl ether, diisopropanolamine, diisopropyl adipate, dimethicone 100, disodium laureth sulfosuccinate, disodium lauryl sulfosuccinate, docusate sodium, edetate disodium, edetic acid, fd&c green no. 3, fd&c yellow no. 6, glycerin, glyceryl oleate, hexylene glycol, high density polyethylene, hyaluronate sodium, hydrochloric acid, hydroxyethyl cellulose, hydroxypropyl cellulose, hypromellose, isopropyl alcohol, isopropyl myristate, isopropyl palmitate, isostearic acid, lactic acid, laureth-4, lavender oil, lecithin, lemon oil, light mineral oil, limonene, mannitol, medium-chain triglycerides, menthol, methyl laurate, methyl salicylate, methylparaben, microcrystalline wax, mineral oil, niacinamide, octoxynol-9, octyldodecanol, oleyl alcohol, parfum creme 45/3, peg/ppg-18/18 dimethicone, peg-hydrogenated castor oil, peg-7 methyl ether, pentadecalactone, phenonip, phenoxyethanol, phosphoric acid, poloxamer, polycarbophil, polyethylene glycol, polyoxyl 20 cetostearyl ether, polypropylene glycol, polysorbate, potassium hydroxide, ppg-15 stearyl ether, ppg-20 methyl glucose ether distearate, propyl gallate, propylene glycol, propylene glycol monolaurate, propylparaben, ricinoleic acid, sodium saccharin, sd alcohol 40-2, sepineo p 600, silicon dioxide, sodium benzoate, sodium chloride, sodium hydroxide, sodium lactate, sodium lauryl sulfate, sodium phosphate, sorbic acid, sorbitan monolaurate, succinic acid, tert-butyl alcohol, titanium dioxide, trisodium citrate dihydrate, trolamine, tromethamine, tyloxapol, xanthan gum, ethanol, butylated hydroxytoluene, carbomer copolymer type c (allyl pentaerythritol crosslinked), carbomer homopolymer type c (allyl pentaerythritol crosslinked), isopropyl myristate, oleic acid, propylene glycol, and sodium hydroxide.

In an embodiment of the present invention the pharmaceutical composition is a throat lozenge. Throat lozenges are pharmaceutical compositions designed to dissolve in the mouth cavity, typically by sublingual or buccal administration. In an embodiment of the invention the extract of fenugreek is administered together with bentonite to potentiate the pharmaceutical effect, i.e. to reduce the number of treatments for alleviating the radiation-induced fibrosis.

The pharmaceutical acceptable additives for the throat lozenge may include one or more of the following substances: acacia, acesulfame potassium, citric acid, aspartame, calcium polycarbofil, citric acid monohydrate, dextrates, dextrose, cinnamon flavor, peppermint falvor, hydroxypropyl cellulose, magnesium stearate, maltodextrin, mannitol, methyl salicylate, peppermint oil, potassium bicarbonate, povidone K30, silicon dioxide, sodium alginate, sodium bicarbonate, sodium carbonate, sodium phosphate, sodium stearyl fumarate, pregelatinized starch, sorbitol, sucralose, sucrose, talc, xanthan gum.

Preferably, when bentonite is present in the composition, the mixture of the extract of Trigonella foenum-graecum and the bentonite is present in the same composition. Alternatively, they may be supplied in a kit of parts, i.e. one part comprises the extract of Trigonella foenum-graecum and the other part comprises the bentonite.

According to the present invention the amount of fenugreek extract is preferably present in “a pharmaceutical effective dosage” of the composition. A pharmaceutical effective dosage refers to the amount necessary to induce the desired biological effect on the subject in need of treatment. Furthermore, the expression pharmaceutical effective dosage covers medical effective dosage and cosmetic effective dosage.

The composition according to the present invention may be administrated once or more than once a day, for example it may be administered in the range of 2 to 10 times a day, such as 2 to 7 times, for example 2 to 5 times, such as 2 to 4 times, such as 2 to 3 times a day.

The composition according to the present invention may be administrated to the subject for a period of treatment of one or more than one week such as two weeks, three weeks, four weeks, five weeks, six weeks, seven weeks, eight weeks, nine weeks, ten weeks, or more than ten weeks. In particular, the composition may be administered during the entire period for the radiation therapy. In some instances, it may be suitable to pretreat the patient with the composition before the initiation of the radiation therapy. Thus, the patient may receive a pretreatment with the composition of the invention, 1-14 days prior to the commencement of the radiation therapy. It may also be suitable in some embodiment of the invention to post-treat a patient having received radiation therapy with the composition of the invention. The post-treatment following the radiation therapy typically has a duration of 1 to 30 days depending on the severity of the radiation-induced dermatitis. The treatment with the present composition may be continued beyond 30 days if considered necessary for inhibiting the development radiation-induced fibrosis.

### Radiation Therapy

Patients with cancer often receive external beam ionizing radiation therapy either alone or in combination with surgery and/or chemotherapy. Ionizing radiation induces damage not only in rapidly proliferating tumor cells but also in normal tissue in the radiation field. Much of the immediate effect in response to irradiation of normal tissue is influenced by the radiosensitivity of individual patients.

One important late effect, that is a significant contributor to patient morbidity, is radiation-induced fibrosis (RIF), which may occur in the skin and subcutaneous tissue, lungs, gastrointestinal and genitourinary tracts, as well as any other organs in the treatment field. Radiation injury triggers inflammation and ultimately stimulates transdifferentiation of fibroblasts into myofibroblasts. In addition to their excessive proliferation, these myofibroblasts produce excess collagen and other extracellular matrix (ECM) components, which is compounded by a reduction in remodeling enzymes. Subsequent fibrosis reduces tissue compliance and—in a majority of cancer patients and particularly those with head and neck cancer—causes cosmetic and functional impairment that significantly impacts quality of life.

Radiation-induced fibrosis usually occurs 3-12 months after radiation therapy and progresses over several years. It affects almost every part of the body that is exposed to radiation. The clinical presentation depends on the type of tissue exposed to irradiation. In general, radiation-induced fibrosis may manifest as skin induration (hardening) and thickening, muscle shortening and atrophy, limited joint mobility, lymphedema, mucosal fibrosis, ulceration, fistula, hollow organ stenosis, and pain.

The mechanism of radiation-induced fibrosis starts with an initial injury that incites an acute response that in turn leads to inflammation, followed by fibroblast recruitment and activation with extracellular matrix deposition. Radiation is energy in the form of waves or high-speed particles. The term “ionizing” indicates that said energy is strong enough to displace bound electrons. Ionizing radiation refers to three types of emissions—alpha, beta, and gamma—with therapeutic radiation being predominantly gamma. Radiation injury results from two primary mechanisms: direct DNA damage and the generation of reactive oxygen species. The latter is more prominent in radiation-induced fibrosis and involves the interaction of ionizing radiation with water molecules to form free radicals, including superoxide, hydrogen peroxide, and hydroxyl radical, the latter of which accounts for the majority of the total damage.

Diseases Treated with Radiation Therapy

Throat cancer refers to cancerous tumors that develop in the throat (pharynx), voice box (larynx) tonsils or oropharynx, but can also refer to cancers that start in the oesophagus (food pipe) or thyroid. Some cancers which begin in the throat area, as well as the tongue, salivary glands, sinuses, nose or ear, are classified as head and neck cancers. The throat is a muscular tube that begins behind the nose and ends in your neck. Throat cancer most often begins in the flat cells that line the inside of the throat. The two main types of cancer that are commonly referred to as throat cancers are pharyngeal and laryngeal cancers—cancer of the pharynx and the larynx.

For early-stage throat cancers, radiation therapy may be the only treatment necessary. For more advanced throat cancers, radiation therapy may be combined with chemotherapy or surgery. In very advanced throat cancers, radiation therapy may be used to reduce signs and symptoms and make the patient more comfortable.

Radiation for Breast Cancer with high-energy rays (or particles) destroys cancer cells. Some women with breast cancer will need radiation, in addition to other treatments. Radiation therapy is used in several situations: After breast-conserving surgery (BCS), to help lower the chance that the cancer will come back in the same breast or nearby lymph nodes; after a mastectomy, especially if the cancer was larger than 5 cm (about 2 inches), if cancer is found in many lymph nodes, or if certain surgical margins have cancer such as the skin or muscle; and if cancer has spread to other parts of the body, such as the bones or brain. The main types of radiation therapy that can be used to treat breast cancer are external beam radiation therapy (EBRT) and brachytherapy. In these patients, fibrosis can result in cosmetic changes of the breast but also severe and harmful indurations and limited mobility.

Radiation-induced pulmonary fibrosis is the late manifestation of radiation-induced lung disease and is relatively common following radiotherapy for chest wall or intrathoracic malignancies. Radiation-induced pulmonary fibrosis is typically seen between 6 and 12 months following completion of radiotherapy course and can continue to progress for up to 2 years. Although the majority of patients are asymptomatic, referred symptoms include a persistent dry cough and shortness of breath. The radiation-induced chronic lung injury may evolve to chronic respiratory failure, pulmonary hypertension, or chronic corpulmonale. Ionizing irradiation causes damage to lung epithelium releasing inflammatory mediators that attract inflammatory cells. These in turn secret profibrotic cytokines and chemokines, amplifying the inflammatory response. These profibrotic mediators stimulate fibroblasts to produce extracellular matrix proteins (e.g. collagen) resulting in the excess deposition of these materials. When fibrosis has become established, no treatment is available, other than a follow-up to assess for tumor recurrence.

Radiation therapy is an essential treatment modality for multiple thoracic malignancies and is a standard treatment for patients with non-small cell lung cancer (NSCLC). The most radiosensitive subunit of the lung is the alveolar/capillary complex. Radiotherapy of the thorax is strongly limited by radiation-induced early side effects in the organ like acute radiation pneumonitis which even may cause interruption or premature termination of therapy.

In head and neck cancer patients, radiation-induced fibrosis can occur. In these patients, again, fibrotic side effects can be rather harmful according to the affected site, for example they strongly affect oral mucosae and swallowing and thus adequate food intake.

Overall, these examples show that tissue fibrosis is a severe side effect of radiotherapy strongly affecting therapy success but also quality of life in cancer survivors.

### EXAMPLES

### Example 1

### Preparation of a Gel

1962 g water was measured in a beaker. 8.00 g Polygel HP obtained from 3V SIGMA S.P.A. and slowly poured into the water during agitation. The agitation was continued for about 5 min to obtain a homogeneous dispersion. During mild agitation 20.0 g Versatil PC obtained from Evonik Dr. Straetmans GmbH was added together with 5.00 g extract of fenugreek is prepared in example 3.

During agitation 24% sodium hydroxide was added until a pH between 5.5 and 6.0 was reached. The agitation continued until a homogeneous and clear gel was obtained.

### Example 2

### Preparation of Throat Lozenges

The following components were added to a container during agitation to obtain a powder mixture:

| | | |
|---|---|---|
| Fenugreek powder | 12.0 | kg |
| Bentonite | 6.0 | kg |
| Menthol | 1.33 | kg |
| Micronized silica gel*⁾ | 4.00 | kg |
| Pepermint | 9.50 | kg |
| Magnesium stearate | 11.00 | kg |
| Sorbitol (granular) | 1,000 | kg |

*⁾Syloid AL-1

The power mixture was conveyed to a tablet press and tablets having an average weight of 1044 mg was obtained.

### Example 3

### Preparation of Fenugreek Powder

Preparation of a powder extract from Trigonella foenum-graecum (fenugreek) seeds was performed as follows: 500 g seeds of Trigonella foenum-graecum were soaked in 2.5 l water for approximately 24 hours. Following the pre-soaking the seeds were cooked for 20 minutes and remains of the seeds were removed from the mixture.

The aqueous extract was spray dried for obtaining a powder in accordance with ISO9001:2008. The aqueous extract had a dry matter content of 1.2-2.0% by weight and a temperature of 5° C. In a concentrate heater the extract was heated to a temperature of 62° C. prior to spraying by a centrifugal atomizer (GEA Niro) running at 12.500 rpm. The dryer inlet temperature was 170° C. and the dryer outlet temperature was 87° C. The spray dried powder was post-dried in a fluid bed at a temperature of 24° C. The powder moisture content of the dried product was 3.58% by weight and the size of the particles was mainly in the range of 5-30 μm.

Due to the low dry matter of the feed extract, the bulk density of the powder was low, i.e. in the range of 0.08 to 0.1 kg/l. A higher dry matter content of e.g. 15% by weight might have yielded a higher bulk density and larger particles.

It was noted that during the spray drying a major amount of the sotolon present in the aqueous extract was evaporated, which produced a spray dried product with less sotolon off-flavor.

### Example 4

A male patient diagnosed with throat cancer received radiation therapy. The radiation was applied through the tissue of the throat from outside the body to reach the area of the diseased tissue (external beam radiation).

The radiation was delivered daily, Monday through Friday, for six weeks, i.e. a total of 30 treatment sessions. The weekend breaks allowed the normal cells to recover. However, as a side effect of the radiation therapy, skin injuries developed due to the skin cells not having enough time to recover between treatments.

After the radiation treatment was ended, the patient had open wounds on the skin as well as ulcers on the mucous membrane inside the throat. The production of saliva was almost non-existing, and the patient had very tough mucous in the throat. Consequently, the patient was not able to eat normally and received tube feeding.

The patient received treatment with the gel according to example 1 and the lozenge according to example 2. During a 8-week period, the gel was applied to the wound on the throat skin 3 timed per day, i.e. morning, noon, and evening. Likewise, lozenges were delivered to the patient 3-10 times daily. After 3 weeks the soar was healed. The treatment was continued for further 5 weeks.

In the start of the treatment, it was difficult for the patient to ingest the lozenges due to pain in the throat but during the first 3 days the pain decreased and was not noticed in the rest of the treatment period. Furthermore, it was observed that the saliva production gradually returned during the treatment period. After the treatment period, the patient was able to eat and drink normally.

3 months after the end of the radiation therapy, the presence of fibrosis tissue was evaluated in the skin of the throat as well as the mucous membrane inside the throat. To the surprise of the doctors, it was not possible to detect any fibrosis tissue on the throat or damages on the mucous membrane of the throat.

The above treatment was repeated on another patient and a similar result was obtained, i.e. fibrosis tissue on the throat skin or damages on the mucous membranes inside the throat could not be detected 3 months after the radiation therapy.

### Example 5

### Preventive Treatment

A female patient diagnosed with throat cancer received radiation therapy. The radiation was applied through the tissue of the throat from outside the body to the reach area of the diseased tissue (external beam radiation).

The radiation was delivered daily, Monday through Friday, for six weeks, i.e. a total of 30 treatment sessions. About halfway through the radiation therapy the patient was treated with the gel according to example 1 and the lozenge according to example 2 in the same way as described in example 4.

As a side effect of the radiation therapy, skin changes developed due to the skin cells not having enough time to recover between treatments. However, the severity of the skin injury on the throat tissue was lower than observed above in example 4. The treatment with the gel and the lozenge was continued for further 2 weeks.

3 months after the end of the radiation therapy, the presence of fibrosis tissue was evaluated on the skin of the throat as well as the mucous membrane inside the throat. To the surprise of the doctors, it was not possible to detect any fibrosis tissue on the throat or damages on the mucous membrane inside the throat.

### Example 6

### Treatment of Side Effects of Breast Cancer

A female diagnosed with breast cancer received radiation therapy. The radiation was applied through the skin tissue of the breast from outside the body to the reach area of the diseased tissue (external beam radiation).

The radiation was delivered daily, Monday through Friday, for six weeks, i.e. a total of 30 treatment sessions. The weekend breaks allowed the normal cells to recover. The gel according to example 1 was applied to the skin area 3 times per day, i.e. morning, noon, and evening during the entire radiation treatment period.

3 months after the end of the radiation therapy, the presence of fibrosis tissue was evaluated in the skin of the breast. To the surprise of the doctors, it was not possible to detect any fibrosis tissue or damages on the skin.

### Example 7

10 persons of mixed gender and age were enrolled in a clinical trial. All persons were subjected to radiation therapy.

The persons were allowed free access to the gel as prepared in example 1 and the lozenge as prepared in example 2. Thus, the gel and the lozenges were self-administrated according to need.

None of the persons had to stop the treatment due to side effects of the radiation damages. About 60% of the patients used a combination of the gel and the lozenges and 40% used the gel only.

4 persons experienced changes in the skin as 2 persons had changes in the pigments and 2 persons experienced formation of wounds. The wounds on the skin were cured in about 2 weeks after the end of the radiation treatment.

After the end of the treatment the formation of radiation-induced fibrosis were evaluated by a physician. Only one person had radiation-induced fibrosis and the depth were evaluated to be less than 1 mm, i.e. a less severe incident.

1-21. (canceled)

22-56. (canceled)

57. A pharmaceutical composition for use in the treatment or prevention of radiation-induced fibrosis, wherein the composition comprises an extract of Trigonella foenum-graecum and optionally pharmaceutically acceptable additives.

58. The pharmaceutical composition according to claim 57, further comprising bentonite.

59. The pharmaceutical composition according to claim 58, wherein the bentonite comprises 50% by weight or more of smectite.

60. The pharmaceutical composition according to claim 57, comprising 4:10 to by weight dry matter of Trigonella foenum-graecum extract to bentonite.

61. The pharmaceutical composition according to claim 57, wherein the weight of Trigonella foenum-graecum extract is at least 0.01% by weight of the pharmaceutical composition.

62. The pharmaceutical composition according to claim 57, wherein said pharmaceutical composition is formulated as a gel, cream, plaster, spray, ointment, throat lozenge, or tablet.

63. The pharmaceutical composition according to claim 57, wherein the disease treated with radiation therapy is throat cancer, breast cancer, lung cancer, melanoma cancer, testis cancer, or head and neck cancer.

64. The pharmaceutical composition according to claim 57, wherein the Trigonella foenum-graecum extract is spray dried particles.

65. A method of treatment or prevention of radiation-induced fibrosis comprising topical application of Trigonella extract foenum-graecum, such as in the form of a gel, cream, ointment or spray and/or oral administration of Trigonella foenum-graecum extract, such as in the form of a plaster, throat lozenge, or tablet.

66. The method according to claim 65, wherein the treatment or prevention topical application includes application of a 2.5% w/w Trigonella foenum-graecum extract gel, cream, ointment or spray.

67. The method according to claim 65, wherein oral administration includes administration of between 0.8 mg-2.6 mg Trigonella foenum-graecum extract/day.

68. The method according to any of claim 65, further comprising administering bentonite, optionally comprising 50% by weight or more of smectite.

69. The method according to claim 68, wherein 4:10 to 10:4 by weight dry matter of Trigonella foenum-graecum extract to bentonite is administered and/or applied.

70. The method according to claim 65, wherein the radiation therapy includes external beam radiation.

71. The method according to claim 65, wherein the radiation-induced fibrosis preventive treatment is initiated during the radiation therapy.

72. The method according to claim 65, wherein the radiation-induced fibrosis treatment is initiated within two years after the end of the radiation therapy.

73. The method according to claim 65, wherein the radiation-induced fibrosis is dermal fibrosis, and preventive treatment or treatment comprises application of a pharmaceutical composition at least once daily on the skin tissue having received radiation.

74. The method according to claim 65, wherein the duration of the treatment is at least one week.

75. The method according to claim 65, wherein the disease treated with radiation therapy is throat cancer, breast cancer, lung cancer, melanoma cancer, testis cancer, or head and neck cancer.

76. The method according to claim 65, wherein the radiation-induced fibrosis is dermal and/or mucosal fibrosis.
